(19) United States
(12) Patent Application Publication
(10) Pub. No.: US 2015/0183772 A1
(43) Pub. Date: Jul. 2, 2015

(54) ALKYLATION OF AZOLES

(71) Applicant: THE BOARD OF TRUSTEES OF THE UNIVERSITY OF ARKANSAS, Little Rock, AR (US)

(72) Inventors: Matthias C. Mcintosh, Fayetteville, AR (US); Silvana Dormi, Fayetteville, AR (US); Juliette Rivero, Fayetteville, AR (US); Sefat Alwarsh, Fayetteville, AR (US); Kolawole Ayinuola, Fayetteville, AR (US)

(73) Assignee: THE BOARD OF TRUSTEES OF THE UNIVERSITY OF ARKANSAS, Little Rock, AR (US)

(21) Appl. No.: 14/403,727

(22) PCT Filed: May 24, 2013
(86) PCT No.: PCT/US2013/042703

§ 371 Date: Nov. 25, 2014

Related U.S. Application Data
(60) Provisional application No. 61/654,433, filed on Jun. 1, 2012.

(51) Int. Cl. C07D 417/06; C07D 277/64

(52) U.S. Cl. CPC C07D 417/06; C07D 277/64

(57) ABSTRACT

In one aspect, methods for the alkylation of azoles are described herein. In some embodiments, a method for the C2 alkylation of an azole compound comprises providing an azolium salt comprising an N-allyl substituent, reacting the N-allyl azolium salt with an aldehyde to provide a reaction product mixture comprising a ketone and N-allyl-N,X-ketene acetal, and providing the C2-alkylated azole compound by Claisen rearrangement of the N-allyl-N,X-ketene acetal, wherein X is selected from the group consisting of S, N and O.

### RELATED APPLICATION DATA

The present application claims priority under 35 U.S.C. §119(e) to U.S. Provisional Patent Application Ser. No. 61/654,433 filed Jun. 1, 2012, which is hereby incorporated by reference in its entirety.

### STATEMENT OF GOVERNMENT LICENSE RIGHTS

This invention was made with support of the National Science Foundation (NSF) grant number 0911638 and National Institutes of Health (NIH) grant number 8P30GM103450. The United States Government has certain license rights in this invention.

### FIELD

The present invention relates to the alkylation of azoles and, in particular, to methods of azole alkylation under mild reaction conditions.

### BACKGROUND

Azoles include a large class of compounds having a five-membered unsaturated heterocyclic ring comprising at least one heteroatom in addition to nitrogen in a 1,3-relationship, the heteroatom selected from the group consisting of oxygen, sulfur and nitrogen. Azoles find application in a variety of fields, including pharmaceuticals. Azoles, for example, are commonly used in antifungal compositions.

Current synthetic methods for producing azole compounds of various structure suffer several limitations such as the use of strong base, expensive transition metal catalyst, high reaction temperatures, dimerization of reaction intermediates and other reaction conditions that restrict functional group compatibility. The foregoing limitations render synthesis and investigation of new azole compounds expensive and difficult.

### SUMMARY

In one aspect, methods of azole alkylation are described herein which, in some embodiments, can mitigate or overcome one or more disadvantages of current azole synthetic techniques. In some embodiments, for example, methods described herein employ mild reaction conditions of weak base and low reaction temperatures and do not require transition metal catalyst and/or cryogens, thereby permitting the facile synthesis of a variety of azole compounds for incorporation into compositions or derivation into other products, including pharmaceutical products and/or carboxylic acid surrogates.

Methods described herein, in some embodiments, provide for the C2 alkylation of azole compounds. In some embodiments, a method for the C2 alkylation of an azole compound comprises providing an azolium salt comprising an N-allyl substituent, reacting the N-allyl azolium salt with an aldehyde to provide a reaction product mixture comprising a ketone and N-allyl-N,X-ketene acetal and providing the C2-alkylated azole compound by Claisen rearrangement of the N-allyl-N,X-ketene acetal, wherein X is selected from the group consisting of S, N and O. Further, in some embodiments, the N-allyl azolium salt is reacted with the aldehyde in the presence of weak base or in the absence of strong base. Additionally, in some embodiments, the ketone of the reaction product mixture is employed in the generation of an amine, hydroxylamine or hydrazine.

In some embodiments of a method for the C2 alkylation of an azole compound described herein, the regioselectivity of the Claisen rearrangement can be altered to provide a majority of [1,3]-rearrangement product or majority [3,3]-rearrangement product. In some embodiments, altering the regioselectivity of the Claisen rearrangement to provide a majority of [1,3]-rearrangement product comprises selecting the N-allyl substituent of the azolium salt to have one or more radical stabilizing moieties. Conversely, altering the regioselectivity of the Claisen rearrangement to provide a majority of [3,3]-rearrangement product comprises selecting the N-allyl substituent of the azolium salt to be deficient in one or more radical stabilizing moieties. Further, in some embodiments, the regioselectivity of the Claisen rearrangement can be altered by addition of a transition metal complex to the reaction product mixture to provide a majority of [3,3]-rearrangement product.

In some embodiments of C2-alkylation of azole compounds described herein, the N-substituent of the azolium salt is not an allyl group. In such embodiments, a method for C2 alkylation of an azole compound comprises providing an N-substituted azolium salt, reacting the N-substituted azolium salt with an aldehyde to provide a reaction product mixture comprising a ketone and an N-substituted-N,X-ketene acetal and providing the C2-alkylated azole compound by rearrangement of the N-substituted-N,X-ketene acetal, wherein X is selected from the group consisting of S, N and O.

In another aspect, methods for C2′ alkylation of azole compounds are described herein. The C2′ position refers to the non-azole ring carbon directly bonded to the C2 carbon of the azole ring. In some embodiments, a method for C2′ alkylation comprises providing an azolium salt comprising an N-allyl substituent and a C2 alkyl substituent, deprotonating the alkyl substituent at the C2′ position in the presence of weak base to provide an N-allyl-N,X-ketene acetal and providing the C2′-alkylated azole compound by Claisen rearrangement of the N-allyl-N,X-ketene acetal, wherein X is selected from the group consisting of S, N and O.

In another aspect, methods of substituting the N-allylic substituent of azolium salts are described herein. In some embodiments, a method of substituting the N-allylic substituent of an azolium salt comprises providing the N-allyl azolium salt, providing an alkene and administering an alkene cross metathesis with the N-allylic substituent and alkene. In some embodiments, N-allyl azolium salts used in methods of C2 and/or C2′ alkylation described herein are provided substituted N-allylic substituents according to alkene cross metathesis techniques.

These and other embodiments are described in greater detail in the detailed description which follows.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

In the structural formulas provided herein and throughout the present specification, the following terms have the indicated meaning:

The term “optionally substituted” means that the group in question is either unsubstituted or substituted with one or more of the substituents specified. When the groups in question are substituted with more than one substituent, the substituent may be the same or different.

The term “alkyl” as used herein, alone or in combination, refers to a straight or branched chain saturated monovalent hydrocarbon radical. In some embodiments, for example, alkyl groups include, but are not limited to, methyl, ethyl, n-propyl, isopropyl, n-butyl, sec-butyl, isobutyl, tert-butyl, n-pentyl, 2-methylbutyl, 3-methylbutyl, 4-methylpentyl, neopentyl, n-pentyl, n-hexyl, 1,2-dimethylpropyl, 2,2-dimethylpropyl, 1,2,2-trimethylpropyl and the like.

The term “alkenyl” as used herein, alone or in combination, refers to a straight or branched chain monovalent hydrocarbon radical containing at least one carbon-carbon double bond. In some embodiments, for example, alkenyl groups include, but are not limited to, allyl, vinyl, 1-propenyl, 2-propenyl, iso-propenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 2,4-hexadienyl, 5-hexenyl and the like.

The term “cycloalkyl” as used herein, alone or in combination, refers to a non-aromatic monovalent hydrocarbon radical ring having from three to twelve carbon atoms, and optionally with one or more degrees of unsaturation. For example, in some embodiments, cycloalkyl groups include, but are not limited to, cyclopropyl, cyclobutyl, cyclopentyl, cyclohexyl, cyclohexenyl, cycloheptyl, cycloheptenyl, cyclooctyl and the like. The term “heterocyclic” or the twin “heterocyclyl” as used herein, alone or in combination, refers to a three to twelve membered ring having atoms of at least two different elements. For example, a heterocyclic group comprises a hydrocarbon ring containing one or more heteroatomic substitutions selected from the group consisting of N, O and S. A heterocyclic ring may be optionally fused to one or more of another heterocyclic ring(s), cycloalkyl ring(s) and/or aryl groups.

The term “aryl” as used herein refers to a carbocyclic aromatic ring radical or to a aromatic ring system radical. Aryl is also intended to include the partially hydrogenated derivatives of the carbocyclic systems.

The term “heteroaryl” as used herein, alone or in combination, refers to an aromatic ring radical with, for instance, 5 to 7 member atoms or to a aromatic ring system radical with, for instance, from 7 to 18 member atoms containing one or more heteroatoms selected from the group consisting of N, O and S.

The term “alkoxy” as used herein, alone or in combination, refers to the monovalent radical RO—, where R is alkyl or alkenyl defined above. For example, alkoxy groups include, but are not limited to, methoxy, ethoxy, n-propoxy, butoxy, sec-butoxy, tert-butoxy, pentoxy, isopentoxy, hexoxy and the like.

The term “weak base” as used herein, refers to a base that is only partially ionized in aqueous solution. For example, weak bases include, but are not limited to, ammonia, primary amines, secondary amines and tertiary amines.

In one aspect, methods of azole alkylation are described herein which, in some embodiments, can mitigate or overcome one or more disadvantages of current azole synthetic techniques. In some embodiments, for example, methods described herein permit the industrial synthesis of a variety of substituted azoles.

### I. C2 Alkylation of Azole Compounds

Methods described herein, in some embodiments, provide for the C2 alkylation of azole compounds. In some embodiments, a method for the C2 alkylation of an azole compound comprises providing an azolium salt comprising an N-allyl substituent, reacting the N-allyl azolium salt with an aldehyde to provide a reaction product mixture comprising a ketone and N-allyl-N,X-ketene acetal and providing the C2-alkylated azole compound by Claisen rearrangement of the N-allyl-N,X-ketene acetal, wherein X is selected from the group consisting of S, N and O.

In some embodiments, a method for the C2 alkylation of an azole compound described herein proceeds according to Scheme 1.

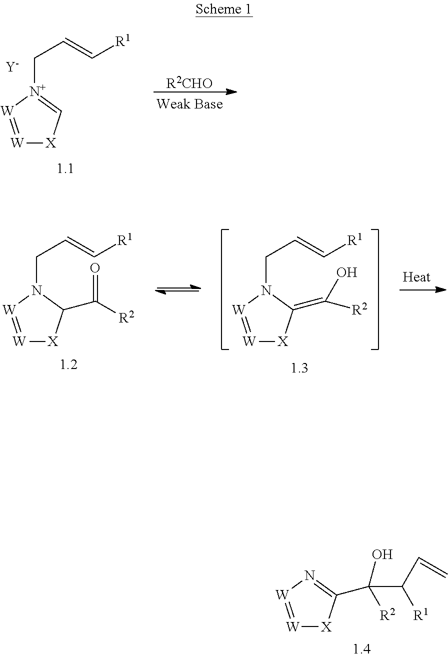

As illustrated in Scheme 1, an azolium salt (1.1) is provided and reacted with aldehyde (R²CHO) in the presence of weak base to provide a reaction product mixture comprising ketone (1.2) and N-allyl-N,X-ketene acetal (1.3). The C2-alkylated azole compound (1.4) is provided by the in situ Claisen rearrangement of the N-allyl-N,X-ketene acetal (1.3). In some embodiments, the Claisen rearrangement is induced by heating the reaction product mixture. Heating the reaction product mixture, in some embodiments, is administered at low temperatures. In one embodiment, for example, the reaction product mixture is heated to a temperature not in excess of 70° C. to provide the C2-alkylated azole compound (1.4) via Claisen rearrangement.

Further, as illustrated in Scheme 1, the azolium salt (1.1) is reacted with the aldehyde under the mild conditions of weak base. As described herein, suitable weak bases can comprise ammonia, 1°-amines, 2°-amines or 3°-amines, other weak bases of similar basicity or mixtures thereof. In one embodiment, for example, Scheme 1 is administered with 1,8-diazabicyclo-[5.4.0]undec-7-ene (DBU). The mild conditions of C2-alkylation synthetic pathways described herein demonstrate compatibility with a variety of functional groups. In the absence of strong base, protecting groups are not required for various functionalities, such as alcohol and amine, in performing the C2 alkylation.

R¹ in Scheme 1, in some embodiments, is selected from the group consisting of hydrogen, alkyl, alkenyl, cycloalkyl, heterocyclyl, aryl and heteroaryl, and R², in some embodiments, is selected from the group consisting of alkyl, alkenyl, cycloalkyl, heterocyclyl, aryl and heteroaryl. X is selected from the group consisting of S, N and O and W is selected from the group consisting of alkyl and N. Additionally, in some embodiments, W is part of a cycloalkyl, heterocyclyl, aryl or heteroaryl ring fused to the azole ring. In one embodiment, for example, W is part of an aryl ring fused to the azole ring to provide benzo-azole structures such as benzothiazole. Moreover, Y⁻ is the counterion for the azolium salt. Y⁻, in some embodiments, is a halide or tosylate.

Scheme 2 illustrates C2 alkylation of an azole compound according to one embodiment described herein. The azolium salt (2.1) of Scheme 2 is benzothiazolium bromide, and the aldehyde is benzaldehyde.

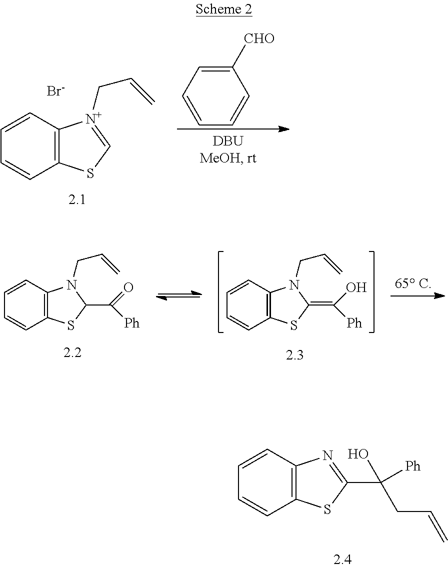

As illustrated in Scheme 2, benzothiazolium bromide (2.1) was reacted with benzaldehyde in the presence of weak base (DBU) to provide a reaction product mixture comprising ketone (2.2) and N-allyl-N,X-ketene acetal (2.3). The C2-alkylated azole compound (2.4) was provided by the in situ Claisen rearrangement of the N-allyl-N,X-ketene acetal (2.3). Additionally, the C2 alkylation of Scheme 2 proceeded without competitive benzoin condensation of the benzaldehyde. In Scheme 2, reaction of benzothiazolium bromide and benzaldehyde was administered for 16 hours and the resulting reaction product mixture was heated at 65° C. for 8 hours to provide a 70% yield of the C2-alkylated azole (14).

Turning now to specific steps, methods for the C2 alkylation of an azole compound described herein comprise providing an azolium salt comprising an N-allyl substituent. Azolium salts suitable for use in methods described herein, in some embodiments, demonstrate a 1,3-relationship between X and nitrogen of the azole ring. In some embodiments, suitable azolium salts comprise oxazoles, thiazoles, imidazoles, 1,2,4-oxadiazoles, 1,2,4-thiadiazoles, 1,2,4-triazoles, 1,3,4-oxadiazoles, 1,3,4-thiadiazoles, 1,3,4-triazoles, 1,2,3,4-oxatriazoles, 1,2,3,4-thiatriazoles and 1,2,3,4-tetrazoles. In some embodiments, for example, suitable azolium salts have any of the following azole ring structures:

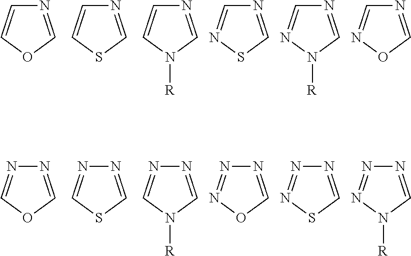

wherein R is selected from the group consisting of hydrogen, alkyl, alkenyl, cycloalkyl, heterocyclyl, aryl and heteroaryl. In some embodiments, the N-allyl substituent of the azolium salt is optionally substituted with one or more species. In some embodiments, the N-allyl substituent is optionally substituted one or more times with a group selected from alkyl, alkenyl, cycloalkyl, heterocyclyl, aryl, heteroaryl and halo. As described further herein, the N-allyl substituent can be substituted with one or more radical stabilizing species. In some embodiments, a variety N-allyl substituents of an azole can be prepared by reaction of the N-allyl substituent with the desired alkene in an alkene cross metathesis reaction.

The N-allyl azolium salt is reacted with an aldehyde (RCHO) to provide a reaction product mixture comprising a ketone and N-allyl-N,X-ketene acetal wherein X is selected from the group consisting of S, N and O. R of the aldehyde can comprise any desirable moiety, such as a saturated or unsaturated hydrocarbon. In some embodiments, the aldehyde moiety (R) is selected from the group consisting of alkyl, alkenyl, cycloalkyl, heterocyclyl, aryl and heteroaryl. In some embodiments, for example, the C2-alkylated azoles (2.7, 2.8, 2.9) were prepared according to Scheme 2 with pyridine-3-carboxyaldehyde, 1-naphthaldehyde and furfural respectively.

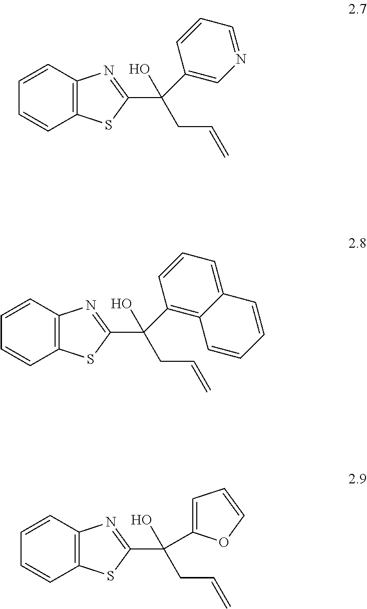 [SMILES: C=CCC(O)(c1cccnc1)c1nc2ccccc2s1 ; C=CCC(O)(c1nc2ccccc2s1)c1cccc2ccccc12 ; C=CCC(O)(c1ccco1)c1nc2ccccc2s1]

The in situ coupling of azole and aldehyde can lend itself to combinatorial synthesis of libraries.

The reaction of the azolium salt and aldehyde is carried out in the presence of weak base. In some embodiments, a weak base comprises ammonia, 1°-amines, 2°-amines or 3°-amines or mixtures thereof. In one embodiment, a weak base comprises DBU. The ability to use weak bases in C2-alkylation methods described herein precludes the use of one or more strong bases. The elimination of strong base increases functional group compatibility of alkylation methods described herein. Additionally, as provided in Scheme 2 above, green solvents including alcohols, such as methanol, can be used in C2-alkylation methods described herein.

The reaction product mixture of ketone and N-allyl-N,X-ketene acetal, in some embodiments, is heated to induce Claisen rearrangement of the ketene acetal to provide the C2-alkylated azole compound. In some embodiments, methods described herein provide low temperatures for the Claisen rearrangement. For example, in some embodiments, the reaction product mixture of ketone and N-allyl-N,X-ketene acetal is heated to a temperature not in excess of 80° C. or 70° C. for Claisen rearrangement. In some embodiment, the reaction product mixture is heated to a temperature ranging from about 55° C. to about 75° C. or from about 60° C. to 70° C. for Claisen rearrangement.

Methods described herein, in some embodiments, further comprise altering the regioselectivity of the Claisen rearrangement to provide a majority of [1,3]-rearrangement product. Altering the regioselectivity of the Claisen rearrangement to provide a majority of [1,3]-rearrangement product, in some embodiments, comprises selecting the N-allyl substituent of the azolium salt to have one or more radical stabilizing moieties. Radical stabilizing moieties of the N-allyl substituent can include aryl, heteroaryl, alkenyl, alkoxy, amino and thio moieties. In addition to selecting the N-allyl substituent to have one or more radical stabilizing moieties, the regioselectivity can be altered to provide a majority of [1,3]-rearrangement product by increasing the reaction temperature of the Claisen rearrangement. In some embodiments, for example, the reaction mixture of ketone and N-allyl-N,X-ketene acetal is heated to a temperature in excess of 60° C. Scheme 3 illustrates altering the regioselectivity of the Claisen rearrangement to provide a majority of [1,3]-rearrangement product according to one embodiment described herein.

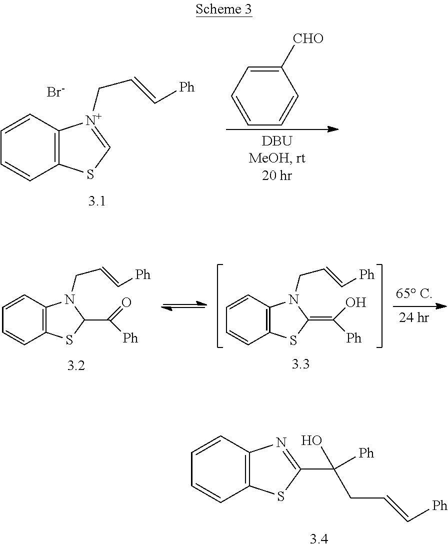

As illustrated in Scheme 3, an N-allyl substituent comprising an aryl radical stabilizing moiety was selected, and the reaction product mixture of ketone (3.2) and N-allyl-N,X-ketene acetal (3.3) was heated to a temperature of 65° C. to afford only the [1,3]-rearrangement product (3.4).

Alternatively, methods described herein, in some embodiments, further comprise altering the regioselectivity of the Claisen rearrangement to provide a majority of [3,3]-rearrangement product. Altering the regioselectivity of the Claisen rearrangement to provide a majority of [3,3]-rearrangement product, in some embodiments, comprises selecting the N-allyl substituent of the azolium salt to be deficient in one or more radical stabilizing moieties. In some embodiments, for example, an N-allyl substituent is selected having only alkyl or cycloalkyl substituents. In addition to selecting the N-allyl substituent to be deficient in one or more radical stabilizing moieties, the regioselectivity can be altered to provide a majority of [3,3]-rearrangement product by lowering the reaction temperature of the Claisen rearrangement. In some embodiments, for example, the reaction mixture of ketone and N-allyl-N,X-ketene acetal is heated to a temperature not in excess of about 60° C. Scheme 4 illustrates altering the regioselectivity of the Claisen rearrangement to provide a majority of [3,3]-rearrangement product according to one embodiment described herein.

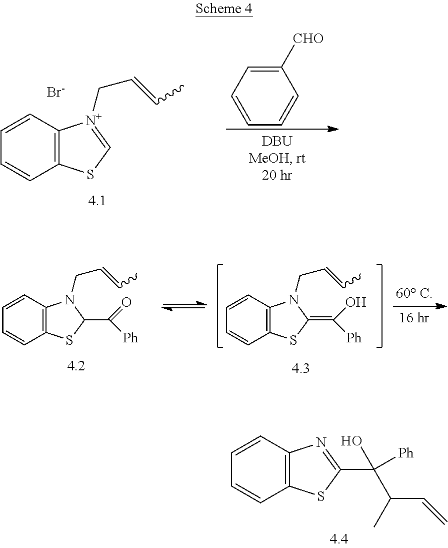

As illustrated in Scheme 4, an N-allyl substituent comprising a crotyl moiety was selected, and the reaction product mixture of ketone (4.2) and N-allyl-N,X-ketene acetal (4.3) was heated to a temperature of 60° C. to afford only the [3,3]-rearrangement product (4.4).

Further, in some embodiments, altering the regioselectivity of the Claisen rearrangement to provide a majority of [3,3]-rearrangement product comprises adding a transition metal complex or other catalyst to the reaction product mixture of the ketone and N-allyl-N,X-ketene acetal. The transition metal complex, in some embodiments, is operable to catalyze the formation of the [3,3]-rearrangement product. Suitable transition metal complexes, in some embodiments, comprise titanium complexes, such as Ti(OiPr)₄. Scheme 5 illustrates altering the regioselectivity of the Claisen rearrangement to provide a majority of [3,3]-rearrangement product with a titanium complex.

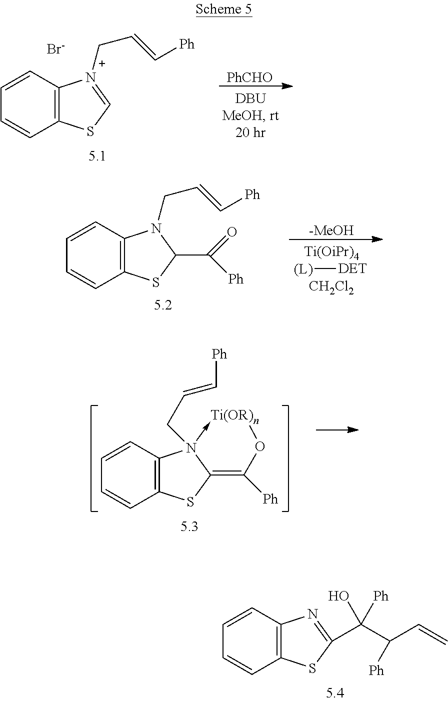

As illustrated in FIG. 5, Ti(OiPr)₄ is added to the reaction product mixture to provide the [3,3]-rearrangement product. The Claisen rearrangement of Scheme 5 to provide the [3,3]-product occurred over three days at ambient temperature. Further, the [3,3]-rearrangement product resulted notwithstanding the presence of an N-allyl substituent having a radical stabilizing aryl moiety.

Additionally, in some embodiments, the ketone of the reaction product mixture is employed in the generation of an amine, hydroxylamine or hydrazine. Ketone formation, in some embodiments, is significantly faster than that of the N-allyl-N,X-ketene acetal and subsequent Claisen rearrangement. Therefore, ketone species in methods described herein, in some embodiments, enable the in situ generation of imines, oximes and hydrazones to provide amines, hydroxyl amines and/or hydrazines. Scheme 6 illustrates formation of amines, hydroxyl amines and/or hydrazines from ketone species of the reaction product mixture according to one embodiment described herein.

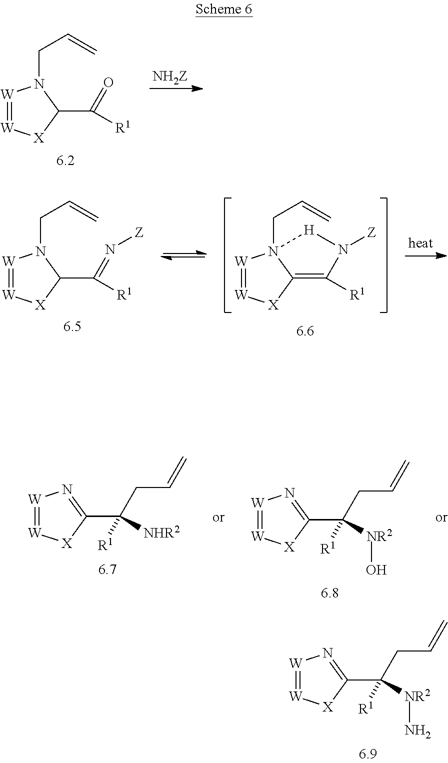

As illustrated in Scheme 6, the ketone product (6.2) is reacted with ammonia or an amine (Z is H or R²) to provide the imine (6.5) and/or enamine (6.6). The amine (6.7), hydroxyl amine (6.8) or hydrazine (6.9) can be subsequently formed. In Scheme 6, R¹ is selected from the group consisting of alkyl, alkenyl, cycloalkyl, heterocyclyl, aryl and heteroaryl, and R² is selected from the group consisting of alkyl and alkenyl. W and X are the same as defined in Scheme 1 herein.

In some embodiments of C2-alkylation of azole compounds described herein, the N-substituent of the azolium salt is not an allyl group. In such embodiments, a method for C2 alkylation of an azole compound comprises providing an N-substituted azolium salt, reacting the N-substituted azolium salt with an aldehyde to provide a reaction product mixture comprising a ketone and an N-substituted-N,X-ketene acetal and providing the C2-alkylated azole compound by rearrangement of the N-substituted-N,X-ketene acetal, wherein X is selected from the group consisting of S, N and O. In some embodiments, for example, the N-substituent is —CH₂Z, wherein Z is selected from the group consisting of aryl, heteroaryl, alkenyl, alkoxy, NR¹R² and SR³, wherein R¹, R² and R³ are independently selected from the group consisting of hydrogen, alkyl, alkenyl, cycloalkyl, heterocyclyl, aryl and heteroaryl.

Scheme 7 illustrates C2 alkylation of an azole compound with an N-substituted azolium salt according to one embodiment described herein.

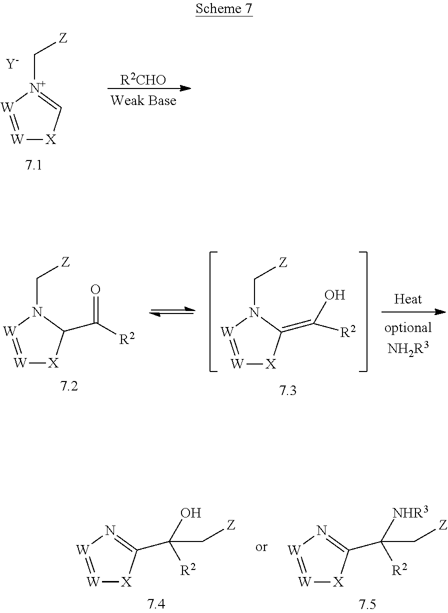

As illustrated in Scheme 7, an N-substituted azolium salt (7.1) is reacted with an aldehyde (R²CHO) in the presence of weak base (e.g. DBU) to provide a reaction product mixture comprising ketone (7.2) and N-substituted-N,X-ketene acetal (7.3). The C2-alkylated azole compound (7.4) is provided by the in situ rearrangement of the N-substituted-N,X-ketene acetal (7.3). Similarly, the C2-alkylated azole compound (7.5) is provided by the in situ Claisen rearrangement of the N-substituted-N,X-ketene acetal (7.3) wherein the hydroxyl has been substituted with amine (NHR³) prior to heating.

In some embodiments, the rearrangement is induced by heating the reaction product mixture. Heating the reaction product mixture, in some embodiments, is administered at low temperatures. In one embodiment, for example, the reaction product mixture is heated to a temperature not in excess of 70° C. to provide the C2-alkylated azole compounds (7.4, 7.5) via rearrangement. In some embodiments, for example, the reaction product mixture is heated to a temperature of 55-65° C. to provide the C2-alkylated azole compounds (7.4, 7.5) via rearrangement.

R² and R³ in Scheme 7, in some embodiments, are independently selected from the group consisting of alkyl, alkenyl, cycloalkyl, heterocyclyl, aryl and heteroaryl. X is selected from the group consisting of S, N and O and W is selected from the group consisting of alkyl and N. Additionally, in some embodiments, W is part of a cycloalkyl, heterocyclyl, aryl or heteroaryl ring fused to the azole ring. In one embodiment, for example, W is part of an aryl ring fused to the azole ring to provide benzo-azole structures such as benzothiazole. Moreover, Y⁻ is the counterion for the azolium salt. Y⁻, in some embodiments, is a halide or tosylate.

### II. C2′ Alkylation of Azole Compounds

In another aspect, methods for C2′ alkylation of azole compounds are described herein. In some embodiments, a method for C2′ alkylation of an azole compound comprises providing an azolium salt comprising an N-allyl substituent and a C2 alkyl substituent, deprotonating the alkyl substituent at the C2′ position in the presence of weak base to provide an N-allyl-N,X-ketene acetal and providing the C2′-alkylated azole compound by Claisen rearrangement of the N-allyl-N,X-ketene acetal, wherein X is selected from the group consisting of S, N and O.

Scheme 8 illustrates C2′ alkylation of an azole compound according to one embodiment of a method described herein.

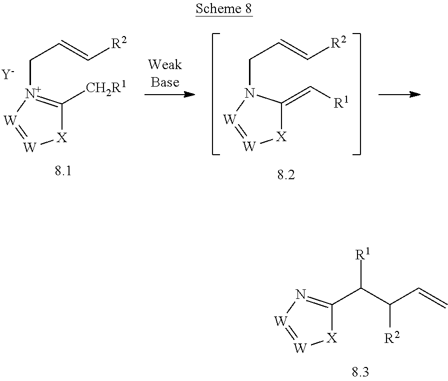

As illustrated in Scheme 8, the C2 alkyl substituent of the azolium salt (8.1) is deprotonated at the C2′ position in the presence of a weak base to provide an N-allyl-N,X-ketene acetal (8.2). The ketene acetal (8.2) undergoes Claisen rearrangement to provide the azole compound alkylated at the C2′ position (8.3). In some embodiments, the ketene acetal (8.2) is heated to induce the Claisen rearrangement. R¹, in some embodiments, is selected from the group consisting of hydrogen and alkyl and hydroxyl, and R², in some embodiments, is selected from the group consisting of hydrogen, alkyl, alkenyl, cycloalkyl, heterocyclyl, aryl, heteroaryl and halo. W, X and Y⁻ are defined in accordance with Scheme 1 hereinabove. In some embodiments, weak base operable for the deprotonation of the alkyl substituent at the C2′ position comprises any of the weak bases described in Section I hereinabove. In one embodiment, for example, a weak base is tetramethyl guanidine (TMG).

Scheme 9 illustrates C2′ alkylation of an azole compound according to some embodiments of methods described herein.

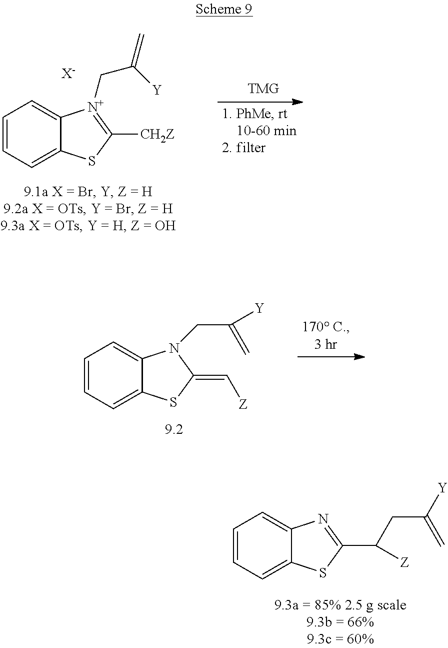

As illustrated in Scheme 9, deprotonation at the C2′ position occurred at room temperature in the presence of TMG to provide the ketene acetal (9.2). TMG tosylate was removed from the reaction product mixture by filtration with a glass frit. Heating the toluene solution of ketene acetal (9.2) induced the Claisen arrangement to provide the C2′ alkylated azole in good yield. Notably, rearrangement of the 2-bromoallyl azolium salt (9.2a) proceeded smoothly to provide bromoalkene, and rearrangement of the unprotected hydroxyl azolium salt (9.3a) also proceeded in satisfactory yield. Further, only the [3,3]-rearrangement product was produced.

III. N-Allylic Substituent Modification

In another aspect, methods of substituting the N-allylic substituent of an azolium salt are described herein. In some embodiments, a method of substituting the N-allylic substituent of an azolium salt comprises providing the N-allyl azolium salt, providing an alkene and administering an alkene cross metathesis with the N-allylic substituent and alkene. In some embodiments, N-allyl azolium salts used in methods of C2 and/or C2′ alkylation described herein are provided substituted N-allylic substituents according to alkene cross metathesis techniques.

Scheme 10 illustrates pathways for substitution of an N-allylic substituent of an azolium salt via alkene cross metathesis according to some embodiments described herein.

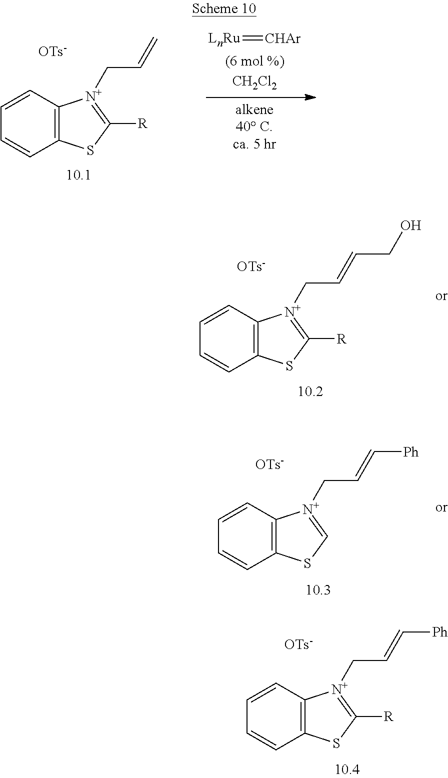

As illustrated in Scheme 10, azolium salts having various N-allyl substituents (10.2, 10.3, 10.4) are provided from a common precursor by the cross alkene metathesis. In some embodiments, N-allyl substituents suitable for use in C2 and C2′ alkylation synthetic methods described herein are prepared in accordance with Scheme 10.

These and other embodiments are further illustrated by the following non-limiting examples.

### EXAMPLE 1

C2 alkylations were administered according to a method described herein as illustrated in Scheme 11. As provided in Scheme 11, various aryl aldehydes demonstrated compatibility with the synthetic pathway to provide [1,3]-rearrangement product.

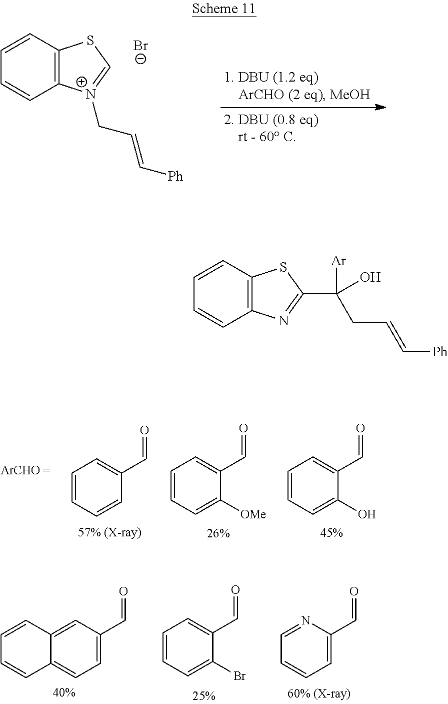

### EXAMPLE 2

A C2 alkylation was administered according to a method described herein as illustrated in Scheme 12 to provide [1,3]-rearrangement product.

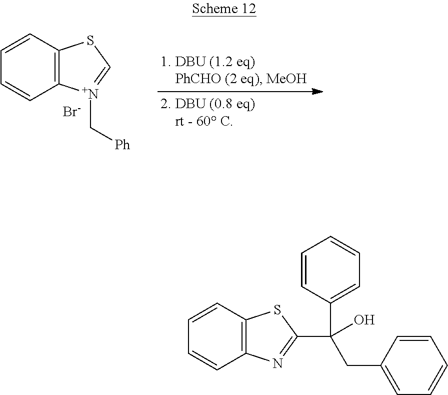 [SMILES: c1ccc(C[n+]2csc3ccccc32)cc1 ; [Br-] ; OC(Cc1ccccc1)(c1ccccc1)c1nc2ccccc2s1]

### EXAMPLE 3

A C2 alkylation was administered according to a method described herein as illustrated in Scheme 13 to provide [1,3]-rearrangement product.

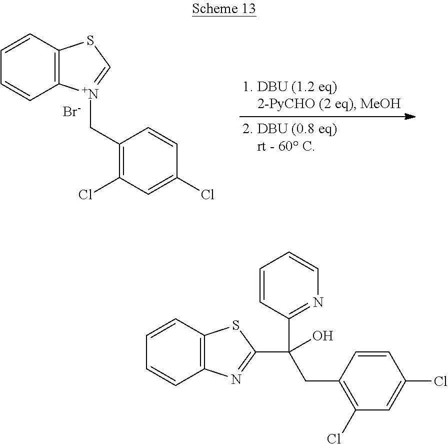 [SMILES: Clc1ccc(C[n+]2csc3ccccc32)c(Cl)c1 ; [Br-] ; OC(Cc1ccc(Cl)cc1Cl)(c1ccccn1)c1nc2ccccc2s1]

### EXAMPLE 4

C2 alkylations were administered according to a method described herein as illustrated in Scheme 14. As provided in Scheme 14, various aryl aldehydes demonstrated compatibility with the synthetic pathway to provide [1,3]-rearrangement product.

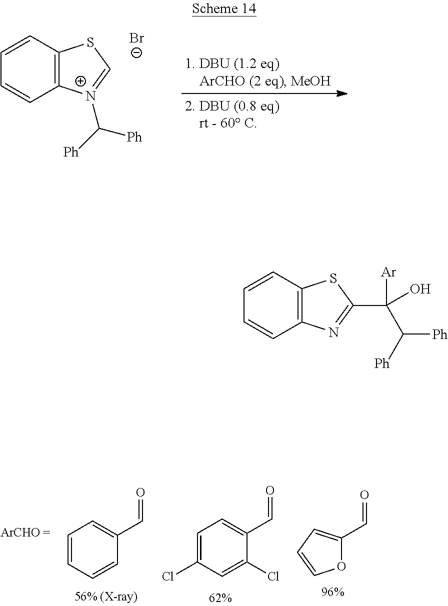

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

That which is claimed is:

1. A method for C2 alkylation of an azole compound comprising:
providing an N-substituted azolium salt;
reacting the N-substituted azolium salt with an aldehyde to provide a reaction product mixture comprising a ketone and an N-substituted-N,X-ketene acetal; and
providing the C2-alkylated azole compound by rearrangement of the N-substituted-N,X-ketene acetal, wherein X is selected from the group consisting of S, N and O.

2. The method of claim 1, wherein the N-substituted azolium salt is reacted with the aldehyde in the presence of weak base.

3. The method of claim 2, wherein the weak base is selected from the group consisting of ammonia, primary amine, secondary amine and tertiary amine

4. The method of claim 3, wherein the weak base is 1,8-diazabicyclo-[5.4.0]undec-7-ene.

5. The method of claim 1, wherein the azolium salt has a 1,3 relationship between X and nitrogen of the azole ring.

6. The method of claim 1, wherein the C2-alkylated azole is a [1,3]-rearrangement product.

7. The method of claim 1, wherein the N-substituent is —CH₂Z and Z is selected from the group consisting of aryl, heteroaryl, alkenyl, alkoxy, NR¹R² and SR³, wherein R¹, R² and R³ are independently selected from the group consisting of hydrogen, alkyl, alkenyl, cycloalkyl, heterocyclyl, aryl and heteroaryl.

8. The method of claim 7, wherein Z is aryl.

9. The method of claim 7, wherein Z is heteroaryl.

10. The method of claims 1, wherein the aldehyde is an unsaturated aldehyde.

11. The method of claim 10, wherein the aldehyde is an aryl aldehyde or heteroaryl aldehyde.

12. The method of claim 1, wherein the azolium salt is a halide or a tosylate.

13. (canceled)

14. The method of claim 1, wherein rearrangement of the N-substituted-N,X-ketene acetal is induced by heating the reaction product mixture.

15. The method of claim 14, wherein the reaction product mixture is heated to a temperature not in excess of 70° C.

16. The method of claim 1, wherein X is S.

17. The method of claim 1, wherein X is O.

18. The method of claim 1, wherein X is N.

19. The method of claim 1, wherein the N-substituted-N,X-ketene acetal is substituted with amine.

20. A method for C2 alkylation of an azole compound comprising:
providing an azolium salt comprising an N-allyl substituent;
reacting the N-allyl azolium salt with an aldehyde to provide a reaction product mixture comprising a ketone and N-allyl-N,X-ketene acetal; and
providing the C2-alkylated azole compound by Claisen rearrangement of the N-allyl-N,X-ketene acetal, wherein X is selected from the group consisting of S, N and O.

21-29. (canceled)

30. The method of claim 20 further comprising altering the regioselectivity of the Claisen rearrangement to provide a majority of [1,3]-rearrangement product.

31-43. (canceled)
